(19) United States
(12) Patent Application Publication
(10) Pub. No.: US 2005/0027174 A1
(43) Pub. Date: Feb. 3, 2005

(54) Methods, systems, and apparatus for monitoring within-day energy balance deviation

(76) Inventor: Dan Benardot, Atlanta, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/903,407

(22) Filed: Jul. 30, 2004

Related U.S. Application Data
(60) Provisional application No. 60/491,927, filed on Aug. 1, 2003.

(51) Int. Cl. A61B 5/00

(52) U.S. Cl. 600/300

(57) ABSTRACT

The invention relates to methods, systems, and apparatus for monitoring within-day energy balance deviation. One aspect of the invention includes a method for automatically determining an energy balance deviation associated with a person that includes providing a device capable of being worn by or accompanying the person, the device adapted to receive information related to the person's energy expenditure, energy intake, and to display energy balance information. The method also includes receiving at least one input associated with energy expenditure of a person, receiving at least one input associated with energy intake of the person, and calculating an energy balance function based in part on the energy expenditure and the energy intake over a period of time. Furthermore, the method includes designating at least one boundary for comparison to said energy balance function, and displaying information corresponding to said energy balance function and said at least one boundary.

### RELATED APPLICATION

This application claims the benefit to U.S. Provisional Application No. 60/491,927 entitled “Methods and Devices for Monitoring Within-Day Energy Balance Deviation,” filed on Aug. 1, 2003, which is hereby incorporated by reference.

### FIELD OF THE INVENTION

The present invention relates to methods, systems, and apparatus for health management monitoring and, more specifically, to health management devices, systems and processes that computationally provide output designed to constantly monitor dynamic within-day energy balance deviations in real time.

### BACKGROUND OF THE INVENTION

The obesity rate in the United States and in other industrialized nations has reached epidemic proportions. It is now estimated by the Centers for Disease Control and Prevention (CDC) that over 20% of the United States Population is obese, and over 55% of the United States Population is overweight (Flegal et al., 1998; National Institute of Health, 1998). See FIG. 1.

As indicated by FIGS. 1 and 2, the prevalence of individual states in this country having populations with aggregate obesity exceeding 15% (but less than 20%) has quadratically increased from seven (7) states to approximately twenty six (26) states during a mere nine year period from 1991 to 2000. A much more troubling statistic is that, in a period of five years beginning in 1995, the number of states in this country having a population with an aggregate obesity exceeding 20% has increased from zero (0) to twenty three (23) states—nearly half of the continental United States.

To further illustrate the rapid proliferation of the health degenerating epidemic of obesity, within the United States nearly 50% of African-American and Mexican-American females meet the Body Mass Index criteria for obesity (Foreyt & Poston, 1998). Body Mass Index (BMI) is a measurement of the relative percentage of fat and muscle mass in the human body, in which weight is divided by height and the result is used as an index of obesity. For example, BMI can be determined using the following formula: BMI=[Weight in pounds/[(Height in inches)*(Height in inches)]]×703. By way of another example, BMI can be determined using the following formula: BMI=[Weight in kilograms/[(Height in meters)*(Height in meters)]]. The NIH and CDC use this index to assess increases in rates of overweight and obesity within segments of relative populations. According to widely accepted standards, “normal weight” is defined as a BMI of 18.5 to 24.9, “overweight” is defined as a BMI of 25 to 29.9, and “obesity” is defined as a BMI of 30 or greater.

The rate of obesity is not gender specific. The NHANES study of 1988-1994 indicated that 27% of females and 22% of males are obese. This study readily reveals that both sexes and all socioeconomic classes have experienced increased rates of obesity. To make matters worse, it is very likely that the actual obesity rate in the U.S. is underestimated by most population surveys because overweight people tend to under-report weight and over-report height.

Furthermore, the increasing trend of obesity is not limited to any segments of the population within the United States. The obesity rates are 24.2% for white non-Hispanics, 30.9% for African-American non-Hispanics, 20.6% for Asians, and 30.4% for Hispanics. Asian-American and Hispanic adolescents born in the U.S. are more than twice as likely to become obese as are first generation residents (Popkin & Udry, 1998). Inner-city populations appear to be at especially high risk for developing obesity. In a cross-sectional survey of inner-city residents of St. Louis and Kansas City, obesity was common (44%) among African-Americans, and many (66%) of the obese were trying (albeit unsuccessfully) to lose weight (Arfken & Houston, 1996). Obesity clearly has reached epidemic proportions, and has become the second (behind smoking) leading cause of preventable death in the United States. Overweight or obese individuals are at increased risk of hypertension, coronary heart disease, certain cancers, type II diabetes and other diseases.

Just as trends of increasing obesity are not limited to groups within the U.S. population, likewise, the rapid increase of aggregate obesity in America is not limited to adults. Approximately 25% of children in the U.S. are overweight, and 30% of childhood obesity cases result in adult obesity (Dietz, 1993). The prevalence of obesity in children increased from 12% in 1991 to 18% in 1998, and these increases were seen in both sexes and all socioeconomic classes (Mokdad et al., 1999). There is no question that public health programs that target children to prevent obesity are important (Gunnell et al., 1998). The concern over childhood obesity rates has so significantly heightened in national priority that Donna E. Shalala, former Secretary of the United States Department of Health and Human Services, announced (June 2000) the release of new CDC pediatric growth charts that now include an assessment of BMI for identifying early weight problems in children. Successful prevention of childhood obesity will involve education programs that encourage appropriate eating and exercise behaviors, that are culturally appropriate, and that can be effectively integrated into families, schools, and communities (Goran et al., 1999).

The genetic pool in the United States has not changed appreciably within the last several decades, during which time the rate of obesity has increased dramatically (Bouchard & Perusse, 1993). Therefore, changes in energy balance, which may result from either excessive energy consumption or reduced energy expenditure are implicated as the primary cause of the sharp rise in the obesity rate rather than genetic predisposition to obesity (Benardot & Thompson, 1999; Hill & Peters, 1998; Jebb, 1999). As a result, countless dietary strategies, ranging from low calorie diets to alterations in the intake of energy substrates (i.e., more protein, less carbohydrate; or less fat, more carbohydrate) have been tried with varying degrees of success in achieving weight loss. Physical activity trials have also demonstrated that exercise is a critically important aspect of weight control and maintenance. However, despite the best intentions of the myriad of available methods of exacting effective weight control, in large part, none of the available dietary strategies have been successful in accomplishing the overriding national objective of reversing the staggering increases of obesity in America.

There are clear indications from past studies that simultaneously modifying both the consumption and expenditure components of the energy balance equation produces the most promising obesity reduction outcomes (ACSM, 1998). Despite these obesity control successes, however, the rate of obesity continues to climb. It is possible that the way energy balance is equated is inaccurate, and there are beginning data suggesting that a more thorough examination of within-day energy balance (as opposed to energy balance calculated in daily or weekly units) may provide important insights into obesity creation (Benardot, 1996; Deutz et al., 2000). Since the cardiovascular disease death rate has been cut during the same two-decade period that obesity has seen a sharp rise, the often-proposed reduction in dietary fat does not appear to have the same beneficial effects on weight that it does in the reduction of cardiovascular disease risk. In fact, there is some evidence that the type of fat consumed (i.e., trans-fatty acids vs. oleic fatty acid) may be more important in obesity control than the proportion of total fat in the diet.

It is likely that obesity rates can be cut in populations following a lifestyle that combines the right dietary modifications and exercise, but it is clear that there are many environmental (i.e., structural) blocks that make the appropriate dietary and exercise changes difficult (Kirk, 1999). One such environmental block to appropriate dietary change is the predominant socialized three-meals-a-day eating pattern in America.

There is an increasing body of evidence suggesting that infrequent eating patterns contribute to the obesity rate. These infrequent eating patterns fail to maintain blood glucose within the normal range (80-120 mg/dl), and cause a catabolism of lean mass, a lowering of metabolic rate, hyperinsulinemia, and a greater fat storage from the consumed foods. In fact, common ‘dieting’ paradigms often result in people missing meals and exacerbating the energy deficits, with outcomes that are counterproductive to the goal of the ‘dieting’. Studies have further shown that it is far more effective for weight loss to consume smaller meals more frequently rather than larger meals less frequently, the latter of which is almost inevitable with the established 3-meal-a-day eating patterns in the United States.

Additional studies specific to athletes have shown that large portions of the athlete community tend to ‘backload’ energy intake. That is, the consumption of calories at the end of the day is extremely high while the intake of energy earlier on in the day is inadequate to meet the energy requirements associated with high levels of physical activity. While this strategy might help athletes achieve an energy balance at the end of the day, it has been demonstrated that this eating behavior creates difficulties in achieving optimal body composition and athletic performance. Within-day energy deficits that occur in athletes have been found to:

Create a poor training benefit

Increase the difficulty of an athlete maintaining existing lean (i.e., muscle) mass

Increase the storage of body fat

Lower metabolic rate (which is associated with decreases in lean body mass)

Diminish the ability of athletes to eat normally without increasing weight (i.e., lower metabolic rates reduce the rate at which calories are burned, making it difficult for athletes to maintain traditional eating patterns without increasing weight.)

Reduce athletic performance (because of less available energy for working muscles)

Increase risk of injury (energy deficits are associated with muscle fatigue and a lower ability to concentrate, both of which are associated with increased risk of injury)

Higher circulating stress hormones (within-day energy deficits may result in low blood sugar, which is inversely related to circulating the stress hormone Cortisol. High Cortisol levels are associated with a catabolism (breakdown) of bone tissue and a reduction of circulating estrogen in females. A common outcome of low estrogen and high Cortisol is lower bone density and an increased risk of stress fracture.

Although there is a need, there are currently no devices, systems or processes available that merge and assess caloric expenditure and caloric intake simultaneously through monitoring physiological and biomechanical values for predicting energy expenditure, and that provide the user with real-time constant monitoring of within-day energy balance deviations.

As evidenced by the obesity statistics in the United States, previous efforts and devices have been ineffective in providing a means by which individuals may actively control their weight and maintain healthy body compositions. The need for devices, systems and processes, according to various embodiments of the present invention, has become more paramount in providing an effective means of curbing the health epidemic of obesity in America.

Given that one of the national health objectives for 2010 is to reduce the prevalence of obesity to less than 15%, a need exists for useful and innovative devices, systems and processes which can assist users in maintenance of healthy body composition by constantly monitoring within-day energy balance deviations and thereby allow the user to stay within a specific desirable energy balance range during the day.

### SUMMARY OF THE INVENTION

Embodiments of the invention provide some or all of the needs described above. One aspect of the invention provides a method for automatically determining an energy balance deviation associated with a person that includes providing a device capable of being worn by or accompanying the person, the device adapted to receive information related to the person's energy expenditure, energy intake, and to display energy balance information. The method also includes receiving at least one input associated with energy expenditure of a person, receiving at least one input associated with energy intake of the person, and calculating an energy balance function based in part on the energy expenditure and the energy intake over a period of time. Furthermore, the method includes designating at least one boundary for comparison to said energy balance function, and displaying information corresponding to said energy balance function and said at least one boundary.

Another aspect of the invention can include an apparatus for monitoring an energy balance deviation associated with a person and capable of being worn by or accompanying the person. The apparatus can include an input component adapted to receive at least one input associated with energy expenditure of a person, and receive an input associated with energy intake of the person. The apparatus can also include a processor adapted to calculate an energy balance function based in part on the energy expenditure and the energy intake over a period of time, designate at least one boundary not to be exceeded by said energy balance function, and display information corresponding to said energy balance function and said at least one boundary.

Another aspect of the invention can include a computer readable medium containing program code adapted to automatically determine an energy balance deviation associated with a person. The computer-readable medium can include program code adapted to provide a device capable of being worn by or accompanying the person to receive information related to the person's energy expenditure, energy intake, and to display energy balance information. Furthermore, the computer-readable medium can include program code adapted to receive at least one input associated with energy expenditure of a person, receive at least one input associated with energy intake of the person, and calculate an energy balance function based in part on the energy expenditure and the energy intake over a period of time. Further, the computer-readable medium can include program code adapted to designate at least one boundary for comparison to said energy balance function, and display information corresponding to said energy balance function and said at least one boundary.

These example embodiments are mentioned not to limit or define the invention, but to provide examples of embodiments of the invention to aid understanding thereof. Example embodiments are discussed in the Detailed Description, and further description of the invention is provided there.

Objects, features and advantages of various systems and processes according to various embodiments of the present invention include:

(1) Devices, systems or processes available that merge and assess caloric expenditure and caloric intake simultaneously through monitoring physiological and biomechanical values for predicting energy expenditure, and that provide the user with real-time constant monitoring of within-day energy balance deviations;

(2) Devices, systems and processes which can assist users in maintenance of healthy body composition by constantly monitoring within-day energy balance deviations and thereby allow the user to stay within a specific desirable energy balance range during the day;

(3) Apparatus, systems, and methods for automatically determining an energy balance deviation associated with a person; and

(4) Apparatus, systems, and methods for monitoring an energy balance deviation associated with a person and capable of being worn by or accompanying the person.

Other objects, features and advantages will become apparent with respect to the remainder of this document.

### BRIEF DESCRIPTION OF THE DRAWINGS

FIG. 1 illustrates obesity trends in the United States.

FIG. 2 illustrates a statistical summary of obesity in the United States.

FIG. 3 illustrates a prediction of a resting energy expenditure based on weight, age, and gender.

FIG. 4 illustrates an estimate of energy expenditure factors for various activities.

FIG. 5 illustrates an example of how to calculate an estimated energy requirement for an active and inactive person.

FIG. 6 is a schematic illustrating a method in accordance with an embodiment of the invention.

FIG. 7 illustrates an example of eating patterns that can affect within-day energy balance.

FIGS. 8A and 8B illustrate an apparatus in accordance with an embodiment of the invention.

FIGS. 9A-9H illustrate an apparatus, process, and various associated screen or graphic presentations for the apparatus and process in accordance with an embodiment of the invention.

FIGS. 10A-10E illustrate another apparatus, process, and various associated screen or graphic presentations for the apparatus and process in accordance with an embodiment of the invention.

FIGS. 11-16 illustrate various screenfaces for a computer-readable medium containing program code for devices, systems and processes according to embodiments of the invention.

FIG. 17 illustrates another apparatus in accordance with an embodiment of the invention.

FIG. 18 illustrates a method that can be implemented by devices, systems, and apparatus in accordance with an embodiment of the invention.

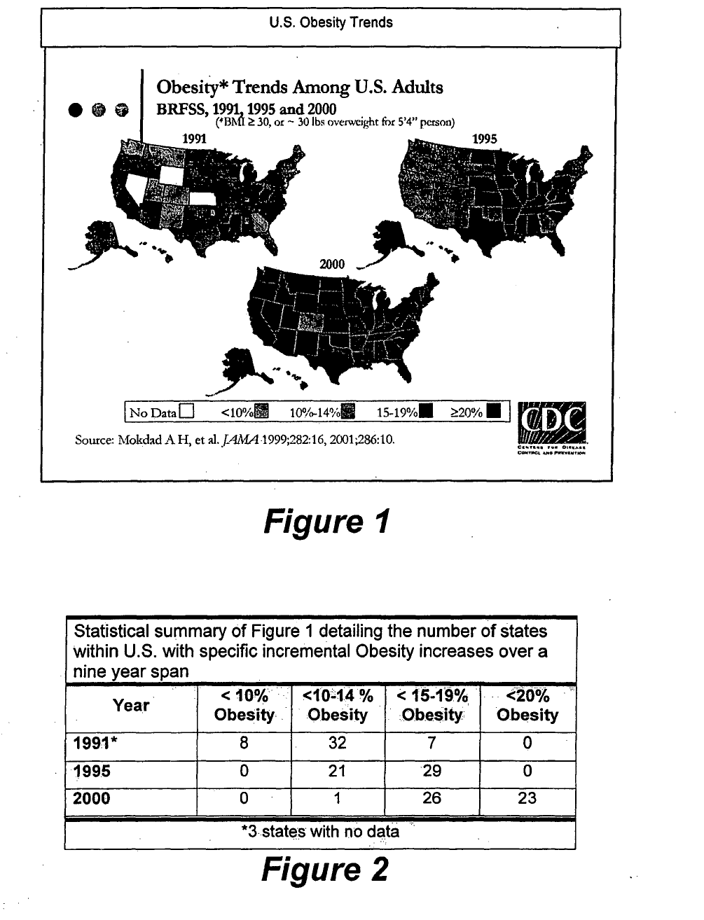

### DETAILED DESCRIPTION OF EMBODIMENTS OF THE INVENTION

Some or all of the above issues, among other things, are addressed by various embodiments of the invention described herein. Various embodiments of the invention can be described with or in conjunction with the following definitions, terms, and associated processes.

Determining Energy (Caloric) Expenditure

Energy expenditure (i.e., the amount of calories expended by a subject over a defined period of time) is a summary value of basal energy expenditure or resting energy expenditure, thermogenesis (resulting from heat loss, the specific dynamic action [SDA] of the diet, and other factors such as drugs), and all physical work beyond the resting state. The SDA of food represents the energy required to extract energy from consumed foods. As an example of the SDA, if twenty (20) calories were required to extract the energy from a piece of fruit from which ninety (90) calories were obtained, then the SDA of consuming the fruit would be twenty (20) calories. This phenomenon of SDA readily parallels the process of lighting a fireplace on a cold winter day. One must first add energy in the form of a lit match, in order to extract a much greater amount of energy in the form of a roaring fire. Basal, or resting, metabolic rate is the main component of energy requirement, accounting for up to 70% of total energy expenditure, thermogenesis counts for approximately 15% of total energy expenditure, and physical activity also accounts for approximately 15% of total energy expenditure.

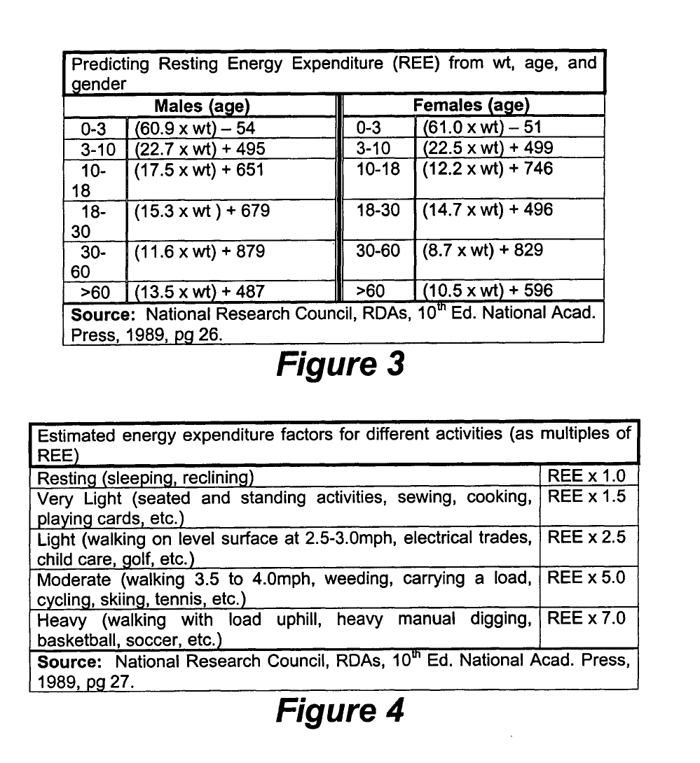

An example of a means of predicting basal or resting energy expenditure is through indirect calorimetry, which assesses oxygen consumed and carbon dioxide expended. However, there are also well-established regression equations for predicting basal or resting energy expenditure from gender, weight, and age (see FIG. 3).

Of these three factors (resting metabolic rate, thermogenesis, and physical activity), only physical activity can vary significantly. Variations in physical activity can result in an energy requirement as little as 5% of total energy requirement in non-athlete populations or as much as 30% of total energy requirement (See FIG. 4) (Ravussin et al., 1986). Alterations in physical activity can help to ‘burn’ excess consumed energy to preserve a lean body composition, but a failure to increase physical activity in the presence of excess consumed energy results in an increased fat storage (Levine et al., 1999). Alterations in physical activity, therefore, are critical in achieving energy balance (Schoeller, 2001).

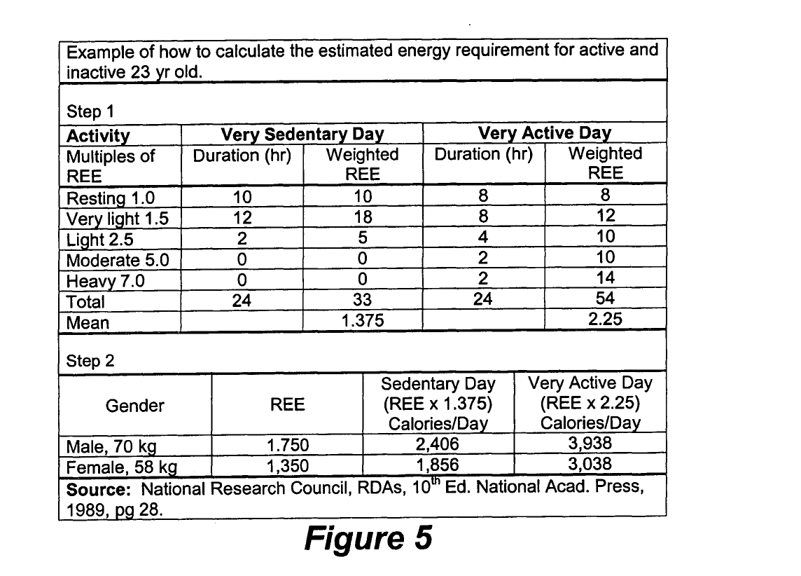

Devices, systems and processes according to certain embodiments of the present invention provide options for entering the basal or resting energy expenditure value that was derived from an indirect calorimetry assessment and/or indirectly by entering gender, height, weight, and age. The calculation of the energy required for different types of physical work of varying durations can be based on MET tables (i.e., tables that provide the metabolic or fractional units above resting energy expenditure of activities that have different exercise intensities), or can be calculated from a regression equation that incorporates body temperature, heart rate, or vertical and horizontal movement velocities. Devices, systems and processes according to certain embodiments of the invention can also use existing and validated technologies that monitor body temperature, heart rate, and movement velocities to predict the caloric cost of physical activity. Accordingly, devices, systems and processes according to various embodiments of this invention can perform some of, all of, and/or more than the following calculations to predict energy expenditure (See FIG. 5).

In the example provided in FIG. 5, a 70 kg (154 lb) male had a predicted caloric requirement of 2,406 calories on a sedentary day and 3,938 calories on a very active day. Caloric requirement was estimated by summing the number of hours in each of five activity levels (resting, very light, etc.) and applying the activity factor to resting energy expenditure. The same procedure was used for predicting the caloric requirement for the female example.

Determining Energy (Caloric) Intake

Energy intake can be predicted, among other ways, from the specific amounts and types of foods consumed over a defined period of time. The basis of the nutrient and caloric content of foods can be determined using freely available computerized databases provided by the United States Department of Agriculture (USDA). The most popular of these databases, USDA Handbook 8—Nutrient Composition of Foods, is periodically updated, with updates made available through the USDA website. Other and/or different sources can be used. A variety of computer software providers have developed software packages that facilitate easy access to this database for the purpose of comparing nutrient and caloric intakes with established standards of intake (i.e., the Food Guide Pyramid, the Recommended Dietary Allowances, etc.). Depending on the goal of the analysis, these databases may provide some of, all of, or more than the amount of information than is needed to estimate caloric intake.

Devices, systems and processes according to certain embodiments of the present invention use any appropriate method for predicting caloric intake, depending on the intended use of the device, system or process. Potential uses and markets for devices, systems, and systems according to certain embodiments of the invention include the entire weight-loss population (and those with obesity-related conditions, such as diabetics); athletes interested in achieving optimal body composition; researchers involved in performance or metabolic studies and health professionals. While many or all of such devices, systems or processes may calculate energy expenditure through the methods described above, they may differ, for example, by the specificity of the food intake modality, the type of activities that may be tracked, and other user or group specific characteristics. Such devices, systems and processes can contain such specialized data and/or programs in memory modules or files which may be inserted in or input into them for various users, groups, and/or markets.

Determining Within-Day Energy Balance

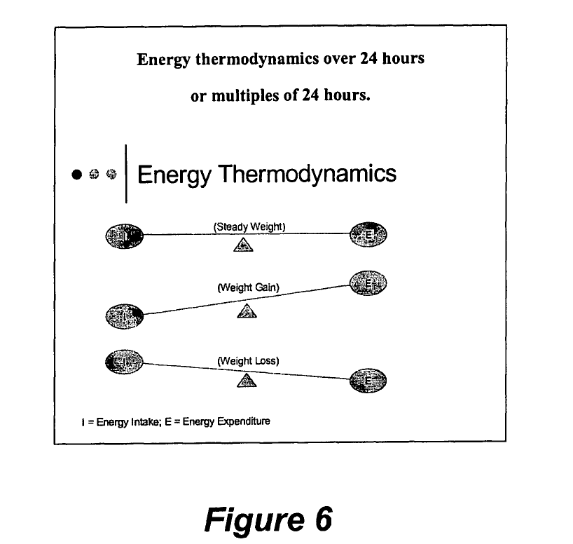

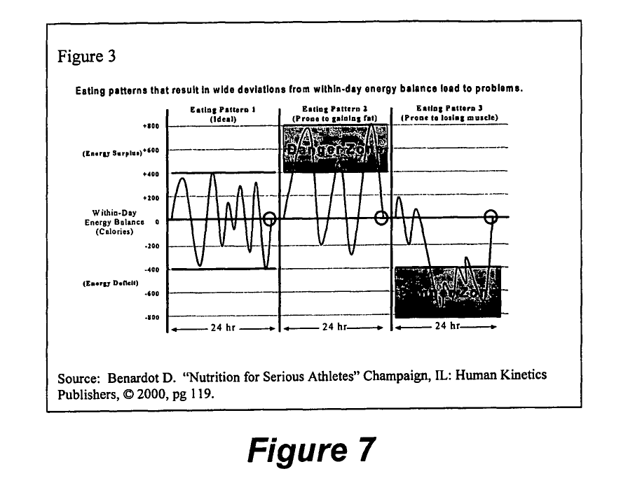

Energy balance can be described as the ratio of energy intake and energy expenditure over a period of 24 hours or multiples of 24 hours. As an example, a person would have a positive energy balance if caloric intake exceeded caloric expenditure; and a negative energy balance if caloric intake were less than caloric expenditure in this time period. This principle of energy thermodynamics (see FIG. 6) has been clearly established, and establishes that a positive energy balance will lead to a weight gain while a negative energy balance will lead to a weight loss.

However, the traditional method of viewing energy balance over periods of 24-hours fails to account for deviations in energy balance that occur during the day. Studies performed by Benardot et al. and published in the scientific literature have demonstrated that the within-day deviations in energy balance are powerful influences on body composition. Devices, systems and processes according to certain embodiments of the invention can therefore preferably constantly monitor within-day deviations in energy balance and provide information to the user to assist her in staying within pre-set energy-balance bounds.

Studies have demonstrated that staying within defined energy-balance bounds during the day results in a maintenance of lean mass, a maintenance of metabolic rate, a lowering of fat mass, and improved nutrient intake (see Eating Pattern 1, FIG. 7). FIG. 7 provides three examples of eating patterns that all result in an energy-balanced state at the end of the day. This is to demonstrate that there are many different ways a person can achieve energy balance, but each way may result in different body composition and performance outcomes. For instance, Eating Pattern 1 represents a person who consumed foods six different times throughout a 24-hour period (6 vertical peaks), and the deviations from perfect energy balance (i.e., deviations from zero {0}) during the day are relatively small. Eating in this manner eliminates the need to eat large amounts at any one time to obtain needed calories, while simultaneously avoiding large energy deficits. Eating Pattern 2, for example, represents a typical 3-meal-a-day eating pattern where large amounts of food must be consumed at each meal to obtain needed calories. This eating pattern creates large energy surpluses that encourage fat storage. Eating Pattern 3 represents the eating behavior often seen in athletes, where much of the day is spent in an energy deficit state (energy expenditure far exceeds intake), but at the end of the day a large meal is consumed to achieve energy balance. While each of these three eating patterns all achieve an energy balance at the end of the day, data are now clearly demonstrating that the magnitude of deficits and surpluses that occur during the day also play an important role in how a person looks and feels. Excessive energy surpluses or deficits during the day increase the risk of obesity (and all the associated disease sequellae, such as diabetes), poor athletic performance, increased injury risk, and poor concentration capacity. Put simply, there are a number of grounds which reinforce the importance of sustaining a within-day energy balance to achieve the desired body composition and to minimize the prevalence of obesity.

Constant monitoring of energy intake and energy expenditure according to certain aspects of the present invention allows for creation of an energy balance ratio in real time, such as, for example, for each minute of the day, and thereby allows the efficient comparison of this ratio to preset energy surplus and deficit parameters. In accordance with the eating patterns described in FIG. 7, devices, systems and processes according to certain embodiments of the invention preferably use a zero-based system (zero being perfect energy balance) to monitor the magnitude of the energy balance deviations away from zero.

Devices, systems and processes according to certain embodiments of the invention can notify users, such as through a series of beeps and/or vibrations, when within-day energy surpluses or energy deficits have exceeded the established bounds for pre-set goals. These cues can advise the user, for example, to eat or stop eating. Additionally, certain embodiments can provide suggestions for what to eat to remain within energy-balance bounds.

As an example of one of many uses contemplated for certain aspects of the invention, a user could begin the day by strapping on a device according to one aspect of the invention shortly after waking up in the morning. The device gives the user a read-out of their current energy-balance so that they can vary the portions of their breakfast according to their actual energy needs, rather than simply eating until they feel “full”. This morning read-out of the user's initial energy balance can be especially important for those users who work out or exercise in the morning. Accordingly, devices, systems and processes according to certain aspects of the invention can allow a user to avoid energy balance deficits (as demonstrated in Eating Pattern 3 of FIG. 7) which may make them prone to losing muscle mass.

As the user progresses through their busy workday, they can effortlessly monitor their within-day energy balance at any given instance. The ability to instantaneously monitor energy balance allows the user to avoid consuming too many donuts or cups of coffee in the office break room because they would be discreetly notified by the device, such as through a series of vibrations and beeps, that preset energy bounds have been exceeded. At lunch, the user would no longer have to speculate aimlessly as to what they should eat. While browsing the menu, the user could simply check their current energy-balance and then press the pre-programmed food-type inputs on the device to determine the foods (and amounts) that would not exceed their caloric intake needs. Likewise, as the end of the workday approaches, because of the device's ability to constantly monitor within-day energy balance, the user could determine whether they were truly in need of a late-afternoon snack to meet their energy needs.

Once home, the user can continue to wear the device until ready to go to bed. Just before going to bed, the user can remove the device and place it in a corresponding recharging cradle which can also automatically synchronize (wirelessly or via hard wire link) the day's incremental energy deficits and surplus with a program in a device such as a personal computer or other device which can also be capable of producing a graphic output. This graphic output can be automatically printed so that, once waking up the next morning, the user has the ability to review their energy-balance patterns of the previous day and thereby become more aware of their personal food consumption habits. The device can therefore allow for more accurate control of the user's caloric consumption based on energy requirements and thereby allow for improved control of body composition and, ultimately, weight.

Devices, systems and processes according to certain embodiments of the invention can therefore improve on existing technologies for predicting energy expenditure and energy intake by uniquely merging them into an integrated functionality that can obtain and/or track dynamic energy balances in real time. In addition, some or all of them can constantly monitor within-day energy balance and alert the user when pre-set energy balance bounds (i.e., excess energy surpluses or deficits) are being exceeded. A primary goal of some such devices, systems and processes is to help the user understand when too many or too few calories are being consumed to satisfy ongoing energy expenditure dynamics. Studies have demonstrated that staying within a narrowly defined caloric buffer which nearly approximates perfect energy balance during the day, will assist in reducing body fat levels. For athletes, staying in energy balance during the day has also been shown to improve athletic performance. Studies have also demonstrated that excess caloric surpluses or deficits during the day are associated with higher body fat levels which, for athletes, can also contribute to poor athletic performance. This is found even when there is an end-of-day energy balance. Devices, systems and processes according to certain embodiments of the invention can help users avoid excess caloric surpluses or deficits and thereby assist them in achieving their desired optimal body composition and/or performance goals.

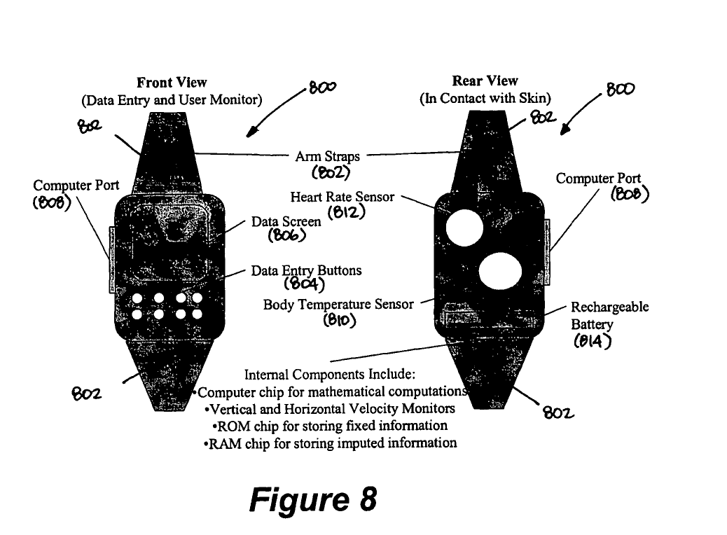

Referring now to the drawings in which like numerals indicate like elements throughout the several figures, the following discussion relates to an apparatus such as a device 800 shown in FIGS. 8A-8B. That device 800 is merely one particular manifestation or embodiment of all the devices, systems, and processes according to the present invention. This device 800 and this discussion therefore does not limit the scope of the invention. Subject to that concern, the device shown in FIGS. 8A and 8B can be a compact self-contained device in the form of a wrist-wearable instrument worn on a person's forearm with an adjustable strap 802 with which the device 800 may be secured to person's wrist or arm, similar to the strap on a wristwatch.

The device 800 can feature a user input interface in the form of buttons 804 that can allow a user to provide the device 800 with data regarding the number of calories in foods consumed by the user. A user input interface can comprise any number of data entry buttons such as 804, a touch screen, a scroll device, or any other suitable, compact means for entering information into the device.

At least one control, such as a button 804, is provided for controlling functions of the energy-balance monitoring device. Various functions can utilize data input from a user via the data entry buttons 804. A display device such as a data screen 806 can display output associated with one or more functions and/or data input from a user.

The device 800 can be designed to be worn by a subject or user during their daily activity to allow convenient and continuous monitoring of their energy balance and caloric consumption and expenditures. The energy-balance monitoring device 800 preferably includes the capability to communicate with local computers by a wired connection, such as a computer port 808, or a wireless means of accomplishing the same, such as by infrared or radio frequency communication. For example, the wristwatch-like embodiment of energy-balance monitoring device 800 can communicate with a local computer by interconnecting a wire between the computer and the device via the computer port 808, or by “docking” the energy-balance monitoring device into a communications “cradle” associated with the computer or personal digital assistance (PDA). It can also communicate using the “Bluetooth” standard, Wi-Fi or any other desired hard or air interface. Similarly, the information from the device 800 can be communicated over the web to a website provided by a service which can assist the user in monitoring their energy balance and diet.

The device 800 can feature one or more sensors for monitoring body temperature and heart rate, such as a body temperature sensor 810 and a heart rate sensor 812. The device 800 can also contain or otherwise include one or more microelectronic motion sensors such as gyros capable of monitoring movement of the device 800 as it is worn by the user.

The device 800 can also contain a microprocessor which can operate as directed by associated software which can be supplied with the device 800 or loaded or upgraded from the internet or as otherwise desired using communication links such as those mentioned above. The processor can comprise a microprocessor, preferably of a type which can be compact and can have very low power consumption. The device can include a rechargeable battery 814 to provide power for the microprocessor and/or other components of the device 800. For example, various processors for use in electronic wristwatches can be used for the processor. The microprocessor can store various user inputs such as calorie consumption information and food content information extracted from downloadable databases. The microprocessor can also extract incremental information collected by the body temperature sensor 810, the heart rate sensor 812, and the food intake to calculate within-day and end-of-day energy balance.

In one embodiment, the microprocessor can generate a visual indicator and can also cause an audible alarm to occur when the user's net calorie consumption or expenditure exceeds the preset within-day energy balance bounds stored in the memory of the device 800. In another embodiment, the microprocessor can generate a notification to occur when the user's net calorie consumption or expenditure exceeds the preset within-day energy balance bounds stored in the memory of the device 800. A notification can be an audible signal, visual signal, tactile signal, or any other type of suitable signal capable of being detected by a user.

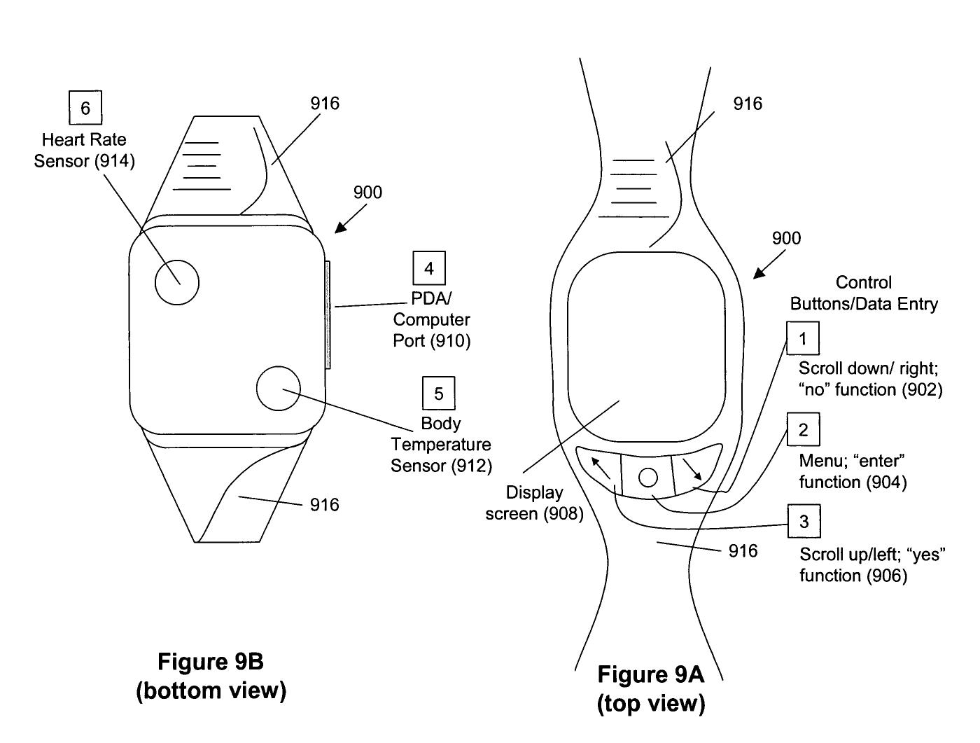

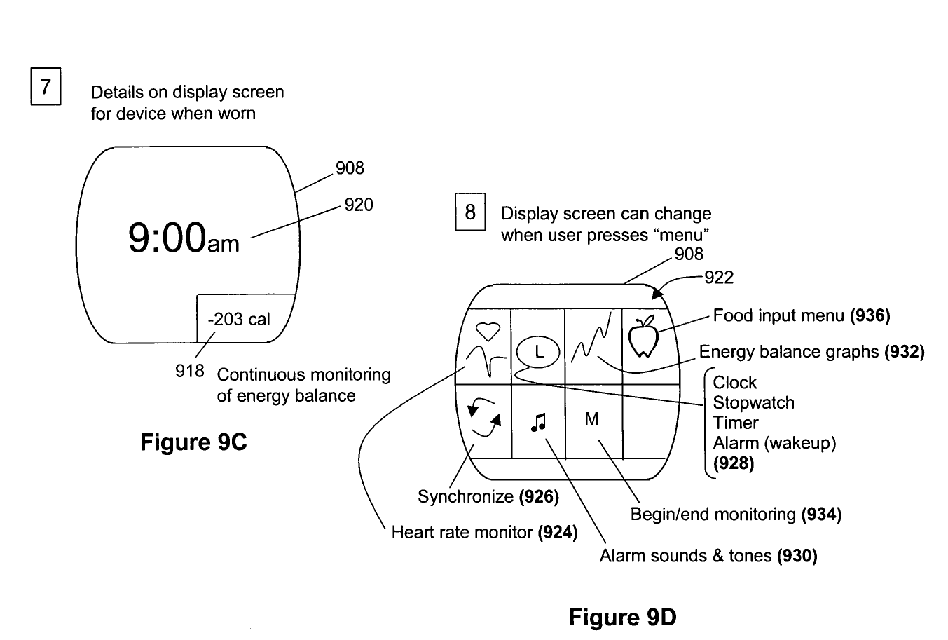

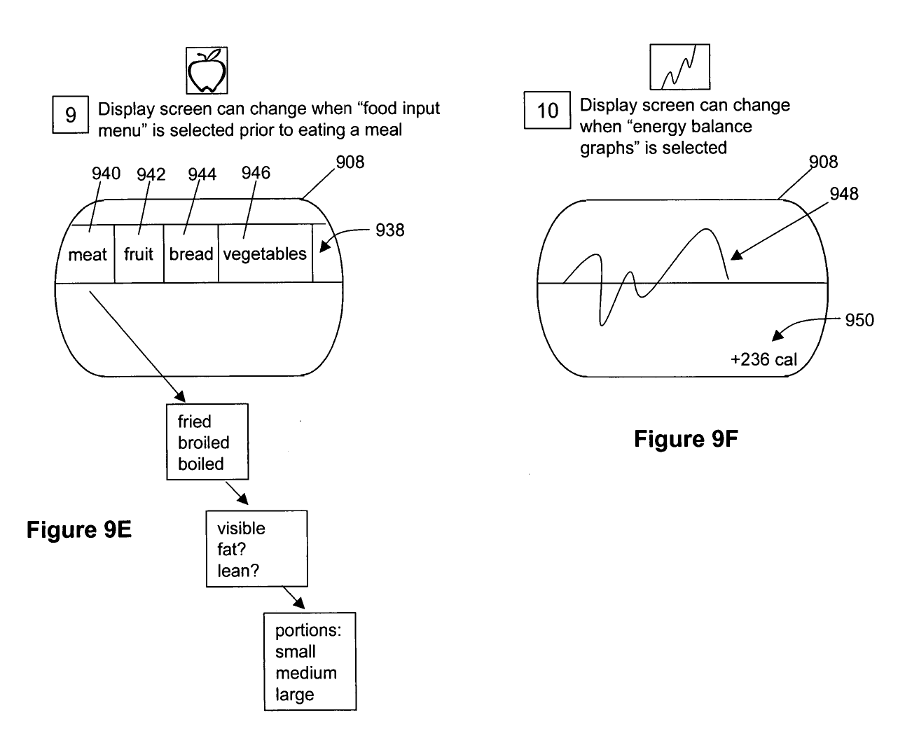

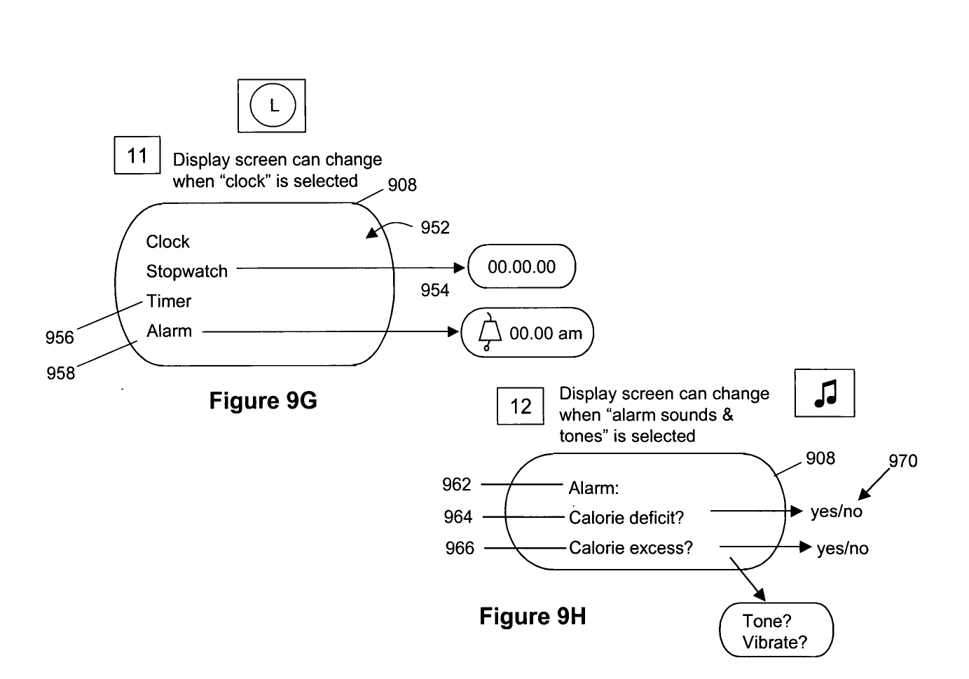

One function of the microprocessor can be to compare the incremental calorie intake and the net calorie intake of the user to the target caloric intake. For example, an “energy balance graph” mode, depicted in FIG. 9F, is a mode which can visually indicate on an associated display device, such as the data screen 806, the mathematical relationship between the cumulative within-day energy balance and the target energy balance.

In one embodiment, the device 800 can sound an audible alarm, or any other means of notification, such as by using a silent vibration mode, when the device 800 first detects that the net within-day energy balance parameters have been reached or exceeded. The device 800 can also trigger an alarm, either silent or audible, whenever a user inputs a food to be consumed by the user in which the number of calories in the food causes the net within-day energy balance to exceed predetermined parameters. In this manner, the device 800 can promote sensible eating since a user can be warned in advance if eating certain food would cause the user to exceed their target within day energy balance.

In one embodiment, the top of the device 900 (the portion that can be viewed, much like the surface of a small PDA) can include a user input interface, such as one or more data entry buttons 804, for entering data such as weight, height, age, and gender that is needed for calculating energy expenditure. Such data can also be loaded into the device 800 via any of the communication links mentioned above. In another embodiment, the device 800 can also include a user input interface, such as additional data entry buttons, for entering the types and amounts of foods consumed (including by the methods of which were described earlier in this document). The device 800 can be light, small, and comfortable to wear, and can present the time of day to obviate the need to wear an additional watch.

FIGS. 9A-9H illustrate another device and an associated process in accordance with an embodiment of the invention. FIG. 9A shows an apparatus such as a wrist-type wearable device 900 similar to the device shown in FIGS. 8A and 8B. FIGS. 9B-9H illustrate various features and associated functionality, further described below, of the device 900 shown in FIG. 9A.

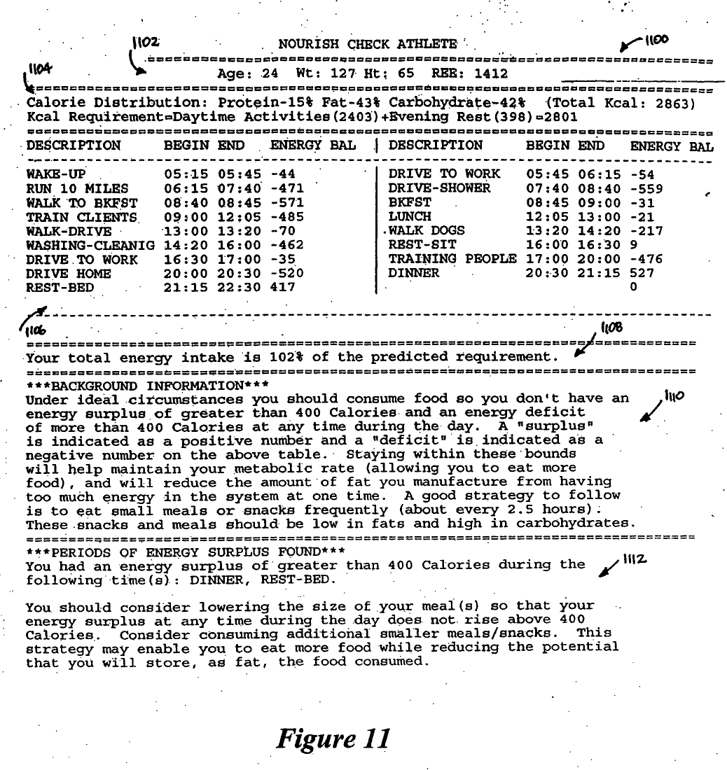

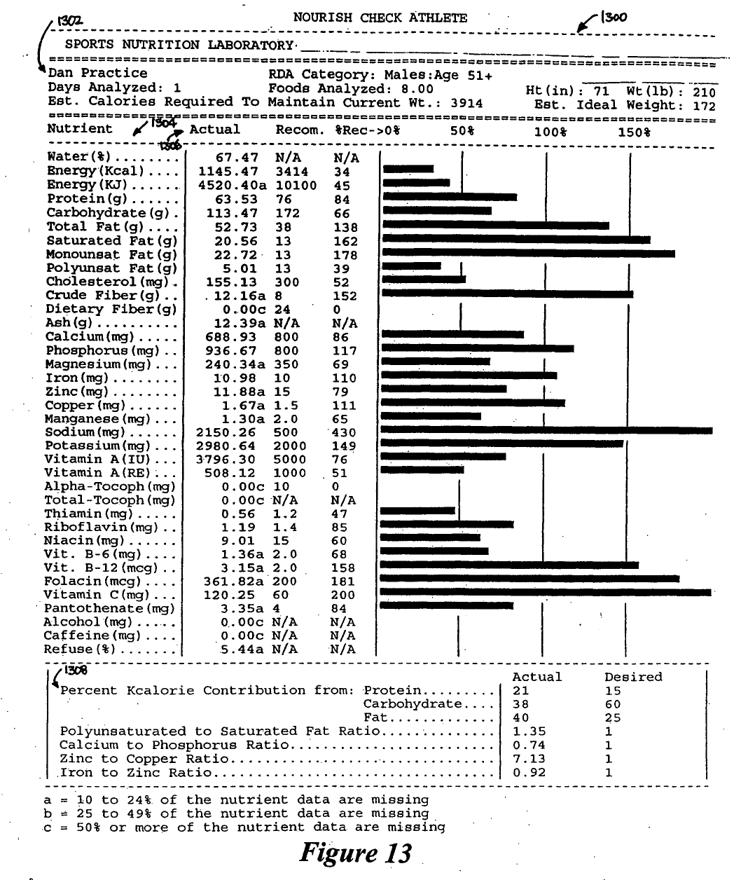

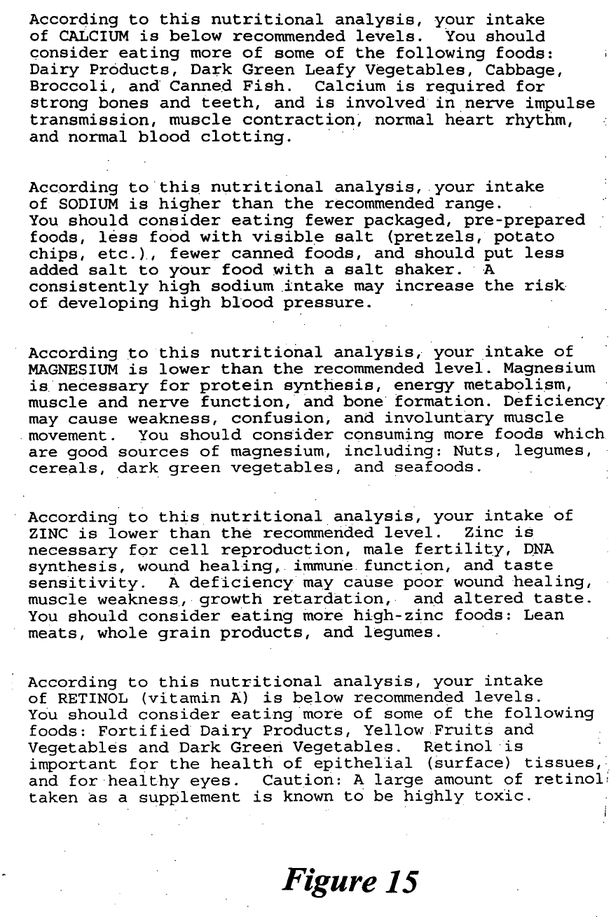

FIGS. 11-15 illustrate examples of screenshots for a remote home personal computer (PC) user input display, according to one embodiment of the present invention. Specifically, FIGS. 11, 12, 13, 14, and 15 depict examples of screenshots for a daily summary of caloric consumption and associated energy balance. These depictions can also provide the user the relative nutrient content of the foods consumed for that period. Additionally, these screenshots for a home PC screen display can provide the background information to help the consumer adjust their eating habits. FIG. 13 depicts an example of a screenshot that displays a more detailed analysis of the relative nutrient content of all the food consumed. These figures are described in greater detail below.

Returning to FIGS. 9A-9H, the device 900 shown can have various features associated with the interfaces shown. As shown in FIG. 9A, the top of the device 900 can feature a user input interface such as three control buttons (1, 2, 3) 902, 904, 906, respectively, which collectively can constitute or otherwise receive a user input. Button (1) 902 can be used to scroll downward or rightward in a menu displayed on an associated display screen 910. In one embodiment, button (1) 902 can be used to provide a “no” indication from a user to a query on the display screen 908. Button (2) 904 can be used to cause an associated microprocessor to change into and out of modes (as shown in FIGS. 9C-9H) in which various parameters associated with the operation of the energy-balance device 900 can be set. Button (2) 904 can also be used to activate various functions such as those shown in FIG. 9D. Button (3) 906 can be used to scroll upward or leftward in a menu displayed on the associated display screen 908. In one embodiment, button (3) 906 can be used to provide a “yes” indication from a user to a query on the display screen 908.

A bottom view of the device 900 is shown in FIG. 9B. Similar to the device shown in FIGS. 8A and 8B, the device 900 can include a PDA/computer port 910, a body temperature sensor 912, and a heart rate sensor 914. The device 900 can be worn via an arm or wrist strap 916, and body sensors 912, 914, similar to 810, 812 respectively, can determine an input associated with energy intake of a user. Fewer or greater body sensors can be used in other embodiments in accordance with the invention. When needed, the device 900 can communicate with a remote computer, PDA, or other processor-based platform via the PDA/computer port 910, similar to the port 808 described in FIGS. 8A and 8B.

The device 900 shown in FIGS. 9A and 9B can include, a microprocessor with associated software or programming to determine one or more energy balance-related functions such as, but not limited to a function of calculating intake of calories, a function of calculating consumption of calories, a function of continuously analyzing within-day energy balance, and a function of simulating predicted energy balance obtained by extrapolating current caloric consumption and future caloric expenditure.

The device 900 can also feature several modes which can be accessed and toggled using the a menu button, such as button (2) 904 in FIG. 9B. In one embodiment, a mode for “continuous monitoring” can be selected and output by the device 900 shown in FIG. 9C. In this particular mode, the device 900 can display an indicator such as a numeric indication that represents continuous monitoring of energy balance for a particular user, such as a user of the device 900. For example, for a particular user, a numeric indication 918 such as “−203 cal” can be output on the display screen 908 for a particular time 920 such as “9:00 am.” In this manner, a user can view the numeric indication or other indicator, and can determine whether his/her energy balance is at or near a particular range or number at a particular time.

In one embodiment, the device 900 can display an input mode menu 922 for selecting at least one of multiple modes. As shown in FIG. 9D, an input mode menu 922 can provide a user with a respective display box 924, 926, 928, 930, 932, 934, 936 for each mode shown on the display screen 908. A user can utilize the buttons 902, 904, 906 to navigate through the input mode menu 922 and select a desired display box 924, 926, 928, 930, 932, 934, 936. Each display box 924, 926, 928, 930, 932, 934, 936 can include text, numbers, an icon, or a combination thereof, such that a user can navigate through the display boxes 924, 926, 928, 930, 932, 934, 936 shown and select a desired display box 924, 926, 928, 930, 932, 934, 936. By way of example only, display box 924 corresponds to a “heart rate monitor” mode for displaying data associated with or otherwise taken by the heart rate sensor 914. This particular display box 924 can include an icon with a heart and a heart rate line. Other display boxes can include other representations corresponding to their respective modes, functionality, or features. Display box 926 corresponds to a “synchronize” mode for synchronizing and/or updating data between the device 900 and another processor-based platform, such as a PDA or computer, via the PDA/computer port 910. Another display box 928 corresponds to a “clock” mode that provides one or more time-related functions such as a clock, stopwatch, timer, and/or alarm (wakeup). Other suitable time-related functions can be provided in accordance with other embodiments of the invention. Furthermore, display box 930 corresponds to an “alarm sounds/tones” mode for providing a user-selection of desired indicators for various functionality such as alarms, warnings, range alerts, or period timers. Display box 932 provides an “energy balance graph” mode for changing and/or selecting a type of graphic user interface for viewing energy balance-related data. Next, display box 934 provides a “begin/end monitoring” mode for providing inputs for a monitoring period associated with an energy-balance calculation. Display box 936 provides a “food input menu” mode for selecting food information for a particular time and/or person, and is described in greater detail below.

Using some or all of the modes and/or functions associated with the display boxes 924, 926, 928, 930, 932, 934, 936 described above, a user can input various selections for food inputs, graphical display options, time-related data, and alarm-related data for an energy-balance monitoring process. An example of an energy-balance monitoring process and respective modes and/or functions associated with such an example are described below.

As an example, the “food input menu” mode can initially be selected by the user, and a “food input” screen can be generated and output by the device 900 as shown in FIG. 9E. In this particular mode, the device 900 can display a detailed food input menu 938 for entering detailed information associated with a food which the user wants to eat. The food information can be input such that a particular food can be selected or otherwise designated from a food list or series of display boxes 940, 942, 944, 946 displayed on the display screen 910 which can be a liquid-crystal display unit. In the displayed food list, a series of display boxes 940, 942, 944, 946 representing one or more respective foods can be classified by types and/or quantities of intake calories. Information about the food to be entered can include type (food group such as “meat,” “fruit,” “bread,” “vegetables;” and fat type such as “visible fat,” “lean”), quantity (detailed or general quantitative measurement such as “small,” “medium,” “large”), and means of preparation of food (example: fried, boiled, broiled, baked, etc). The device 900 can determine or otherwise derive an input associated with energy intake from some or all of the selected food information from the user. In this manner, a user can select one or more particular foods that may be eaten prior to eating a meal comprising such foods. The device 900 can utilize the selected food information to determine an input associated with energy intake associated with the user. The input can be utilized in an energy-balance function, or other function in accordance with embodiments of the invention.

By way of continuing the above example, the “energy balance graph” mode can then be selected for viewing an energy balance graph on the display screen 908 as shown in FIG. 9F. In this particular mode, the device 900 can output an energy balance graph 948 and an indicator such as a numeric indication 950 representing an energy-balance function for a particular time. By way of example, the energy-balance graph 948 shown is a graphical plot of an energy-balance function over time, and the numeric indication 950 shown is a corresponding graphical plot point such as “236 cal” representing an energy-balance function at a particular time. In another embodiment, a user can change and/or select a particular type of graphic user interface for viewing energy balance-related data. For example, other types of graphs or representations of an energy-balance function or suitable energy-balance related data can be output to the display device 908.

By way of continuing the above example, the “clock” mode can then be selected for viewing one or more time-related functions such as a clock, stopwatch, timer, and/or alarm (wakeup) on the display screen 908 as shown in FIG. 9G. In this particular mode, the device 900 can output a clock menu 952 for selecting particular time-related data for monitoring an energy-balance and/or for determining an energy-balance function or similar type function. By way of example, the clock menu 952 shown includes a list of time-related options such as “clock,” “stopwatch,” “timer,” and “alarm.” Selection of the “stopwatch” option 954 can cause a stopwatch-type format 956 such as a time format indication “00.00.00” to be output to the display screen 908 for the user to modify or otherwise enter a period of time to monitor. Furthermore, selection of the “alarm” option 958 can cause an alarm-type format 960 such as “00:00 am” to be output to the display screen 908 for the user to modify or otherwise enter a time for an alarm.

By way of continuing the above example, the “alarm sounds/tones” mode can then be selected for providing a user-selection of desired indicators for various functionality such as alarms, warnings, range alerts, or period timers on the display screen 908 as shown in FIG. 9H. In this particular mode, the device 900 can output an alarm menu 962 for selecting particular alarm-related data for providing an alarm for an energy-balance and/or for providing an alarm for an energy-balance function or similar type function. By way of example, the alarm menu 962 shown includes a list of alarm-related options such as “calorie deficit,” and “calorie excess.” Selection of the “calorie deficit” option 964 can cause a query for an alarm-type format 966 such as a query indication “yes/no” to be output to the display screen 908 for the user to select or otherwise set an alarm associated with a particular calorie deficit. Furthermore, selection of the “calorie excess” option 968 can cause a query for an alarm-type format 970 such as “yes/no” to be output to the display screen 908 for the user to select or otherwise set an alarm associated with a particular calorie excess. In either or both instances, a type of alarm can be set for a particular “calorie deficit” and/or “calorie excess,” such as a particular audible tone or a tactile tone. Some or all of the modes and/or functions described above can be used in an energy-balance monitoring process, and additional modes and/or functions can be implemented in an energy-balance monitoring process in accordance with other embodiments of the invention. An example of another energy-balance process is as follows.

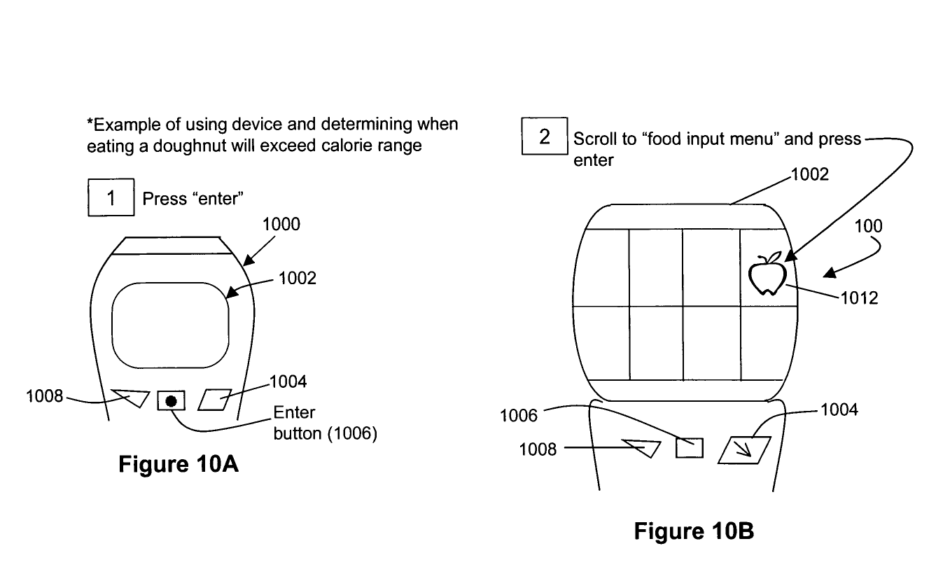

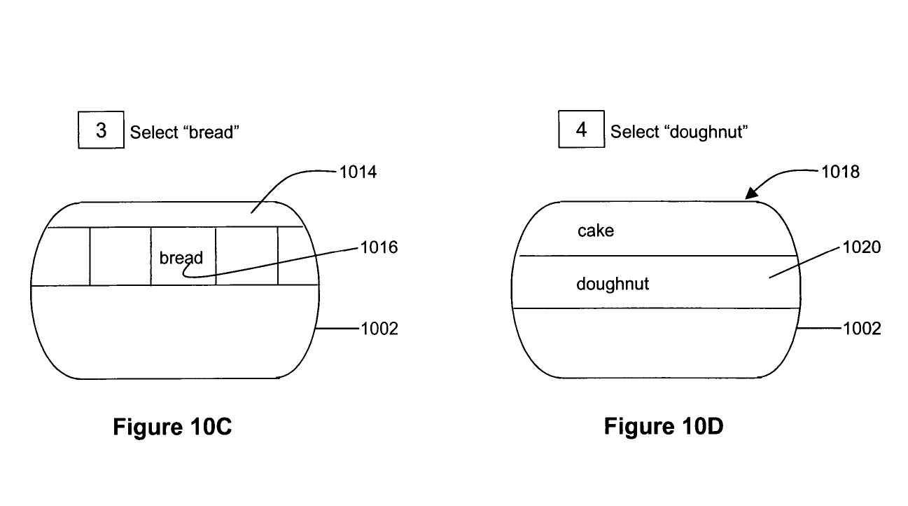

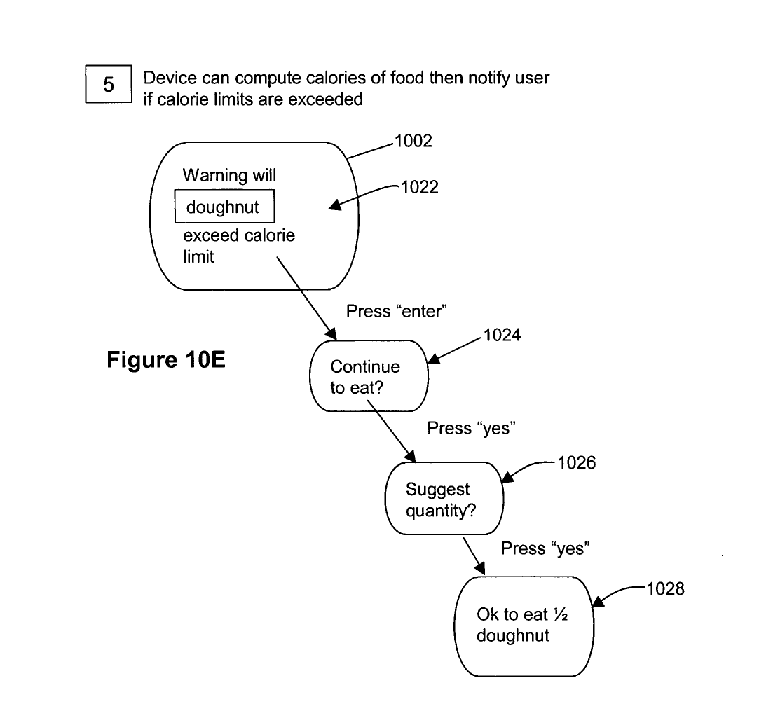

FIGS. 10A-10E illustrate an example in which a user may want to consume a food item such as a doughnut, and the process by which a device such as 1000 determines whether a user's current energy balance can accommodate the calories from the doughnut. This particular process can be implemented alone, or during or in conjunction with the energy-balance monitoring process described above. Some or all of the following modes and/or features can be implemented with the process described below, and other modes and/or features can be implemented with the process as described.

In FIG. 10A, an apparatus such as device 1000 includes a display screen 1002 and one or more data entry buttons 1004, 1006, 1008. Each of the buttons 1004, 1006, 1008 can transmit or otherwise facilitate selection commands from a user such as a wearer of the device 1000. In this example, the centrally-located button 1006 can transmit an “enter” command from a user, and the adjacent buttons 1004, 1008 can transmit movement or navigational commands from the user.

In FIG. 10B, the display screen 1002 includes an output such as a input mode menu 1010, similar to the menu 922 shown and described in FIG. 9D. The user can manipulate one or both adjacent buttons 1004, 1008 to navigate through the input mode menu 1010 to reach a desired display box, such as a display box 1012 with a “food-type” icon representing a corresponding “food input menu” mode. When the user reaches the desired display box, the display box 1012 can be highlighted and the user can then transmit an “enter” command via the centrally-located button 1006 to designate the user's selection of the display box 1012.

In FIG. 10C, the display screen 1002 can output a “detailed food input menu” 1014 similar to the menu 938 shown in FIG. 9E. The user can manipulate one or both adjacent buttons 1004, 1008 to navigate through the menu 1014 to reach a desired display box, such as a display box 1016 with the text “bread” representing a corresponding food type. When the user reaches the desired display box, the display box 1016 can be highlighted and the user can then transmit an “enter” command via the centrally-located button 1006 to designate the user's selection of the display box 1016.

In FIG. 10D, the display screen 1002 can output another “detailed food input menu” 1018 similar to another menu described in FIG. 9E. The user can manipulate one or both adjacent buttons 1004, 1008 to navigate through the menu 1018 to reach a desired display box, such as a display box 1020 with the text “doughnut” representing another corresponding food type. When the user reaches the desired display box, the display box 1020 can be highlighted and the user can then transmit an “enter” command via the centrally-located button 1006 to designate the user's selection of the display box 1020.

In FIG. 10E, the display screen 1002 can output information associated with one or more boundaries, alarms, and/or time-related data. In the example shown, the device 1000 can utilize food information associated with the user's data input from FIGS. 10A-10D to determine an energy input for an energy balance function, such as a caloric intake associated with the selected food item(s). The device 1000 can then determine the energy balance function, and further determine whether the energy input will exceed a boundary set for the energy balance function. Depending on the outcome, the device 1000 can output a message or otherwise prompt the user with one or more information messages 1022, 1024, 1026, 1028 output to the display screen 1002. In this example, the device 1000 can determine that the calories of the doughnut exceed the caloric limit boundary set for a particular energy balance function. The device 1000 can output message 1022 to alert the user via the display screen 1002, such as “Warning, doughnut will exceed calorie limit.” After a predefined amount of time, or upon the user's acknowledgement, such as transmitting an “enter” command via the centrally-located button 1006, the device 1000 can prompt the user with another message 1024 to the display screen 1002, such as “Continue to eat?” The user can respond by entering a response via one or more of the buttons 1004, 1006, 1008, such as entering a “yes” command. Another message 1026 can be output to further prompt the user to input additional information, such as “Suggest quantity?” The user can respond by entering a response via one or more of the buttons 1004, 1006, 1008, such as entering a “yes” command. The device 1000 can then determine an amount of a particular food, such as the doughnut, that the user can eat and stay within the boundaries set for the particular energy balance function. When the device has determined the amount, the device 1000 can output a corresponding message 1028 to the display screen 1002 such as “ok to eat ½ doughnut.” In this manner, the user can utilize a device 1000 in accordance with an embodiment of the invention to input a planned caloric intake prior to a meal, and to determine whether the caloric intake will exceed a boundary set for an associated energy balance function.

In another embodiment, the device 900 shown in FIGS. 9A and 9B can include a docking station capable of recharging its batteries, and also to enable communications with a local computer such as a home PC. When on the docking station the device 900 can download its collected information for additional analysis and printouts (similar to that seen in FIG. 7) of the within-day energy balance variations. The device 900 adapted to be used in clinical use could also include the option of downloading the information to enable a nutrient analysis of foods.

The computer linked to the docking station contains computer software that can include an associated nutrient database for nutrient intake analysis, and corresponding food codes entered in the device 900 while worn for matching the food codes stored in the software to enable a full nutrient intake analysis. The software can analyze and determine, among other things, the number of hours spent in energy surpluses or deficits that exceed the established or predefined bounds or boundaries; and the largest or other predefined quantities or trends associated with energy surpluses and deficits. The software can also compare surpluses and deficits from different days or other periods of analysis, and produce a log of weight changes (and height changes if a child) from different analyses. The software can also produce a description of the periods in the day or another time with the greatest or other quantity or trend of energy expenditures (for future meal planning purposes); a description of the periods in the day with the lowest energy expenditures (for future activity planning purposes) and full nutrient intake analysis that compares the intake of vitamins and minerals to the recommended dietary allowances, with recommendations of what foods to consume for nutrients that are below the recommended levels.

Operations: Options for the General Population

In devices, systems and processes of certain embodiments of the present invention for the general population, energy intake (food consumption) can be estimated through simple push-button descriptions of relative meal size and fat content. The rationale behind this example method is that protein and carbohydrate can provide the same caloric density (4 calories per gram) while fat provides a higher caloric density (9 calories per gram). By identifying foods by their relative fat content and by the amounts consumed, it can be possible to estimate the approximate caloric load of the meal. In addition, this example method is relatively quick, fairly intuitive, and can require relatively little training.

Subjects using this more basic version could wear a device according to an embodiment of the invention on their arm and follow a relatively basic calibration/quality assurance routine that is built into the device. The device can immediately begin recording energy expenditure and can store that information in 15-minute units. When the subject is ready for breakfast, she can enter the relative fat content of the foods to be consumed to give the device an opportunity to provide some guidance on whether the amounts are appropriate for maintaining established energy bounds or predefined boundaries (i.e., the deviations from perfect energy balance, such as ±300 or 400 calories). If the device indicates the selected foods in the amounts indicated are appropriate, it can provide a ‘go ahead signal’. Once the foods are consumed, the user can have an opportunity to adjust the amounts and relative fat level of the foods to accurately record the ‘actual’ amount of food consumed. At around mid-morning or another particular time, it can be possible that the device might trigger an alarm to let the user know that it is time to eat a small snack to avoid going into an excessive energy deficit. In one embodiment, this procedure of recording foods consumed and getting feedback from the model can repeat itself for a predefined period of time, such as a 24 hour time period. At the end of 24 hours or at any other desired or predefined period of time, the user could (optionally) place the model into a receptacle that communicates with a computer to download the information from the previous day. The computer software in the computer that interoperates with the device can provide a graphical display and printout of the energy surpluses and deficits that occurred during the day.

Operation Options for Public Health, Fitness Enthusiasts, and Weight Loss Program Attendees

In devices, systems and processes of other embodiments of the present invention aimed at public health, fitness or weight loss uses, energy intake (food consumption) can be estimated through a pre-entered food list of foods commonly consumed by individual users, or foods recommended by weight loss program. The list can also contain information from the USDA database for the caloric content of foods.

Additional food lists can be available for deviations from the norm, and users can have the option of updating food lists and their caloric content by entering the information from food labels. Most foods can be entered for analysis through pre-set buttons to make for relatively easy and speedy food entry.

Subjects using these sorts of devices can place it on their arm and follow a basic calibration/quality assurance routine that is built into the device. The device can immediately begin recording energy expenditure and can store that information in 15-minute units. When the subject is ready for breakfast, she can select the foods and amounts to be consumed from a database of foods stored in the device. The device can provide guidance on whether the amounts of calories to be consumed are appropriate for maintaining established energy bounds or other predefined boundaries (i.e., the deviations from perfect energy balance, such as ±300 or 400 calories). If the device indicates the selected foods in the amounts indicated are appropriate, it can provide a ‘go ahead signal’. Once the foods are consumed, the subject can have an opportunity to adjust the ‘actual’ types and amounts of food consumed. During the day it can be possible that the device might trigger an alarm to let the subject know that it is time to eat a small snack to avoid going into an excessive energy deficit. In one embodiment, this procedure of recording foods consumed and getting feedback from the device can repeat itself for a predefined period of time, such as a 24 hour period of time. The device can communicate with other computers, websites or other platforms or functionalities as can any other devices, systems or processes according to various embodiments of the invention.

Operation Options for Research and Clinical Settings

In devices, systems and processes of other embodiments of the present invention aimed at research and clinical settings, energy intake (food consumption) can be estimated through built in comprehensive computerized food lists, with multiple options to adjust for food quantity and preparation type. These food lists can contain information from the USDA database for the caloric content of foods, and can also include information from a comprehensive list of nutrients associated with these foods to enable a nutrient (i.e., vitamin and mineral) analysis.

Additional food lists can also be available for deviations from the norm (i.e., Asian foods, etc.) and users can have the option of updating food lists and their caloric/nutrient content by entering the information on food labels. Certain foods could be entered for analysis through a PDA-like interface that allows for food selection from lists, with specific adjustments for amounts consumed.

Subjects using this device can place it on their arm and follow a basic calibration/quality assurance routine that is built into the device. The device immediately can begin recording energy expenditure and can store that information in one-minute units. When the subject is ready for breakfast, she can enter the foods to be consumed to give the device an opportunity to provide some guidance on whether the amounts are appropriate for maintaining established energy bounds or other predefined boundaries (i.e., the deviations from perfect energy balance, such as ±300 or 400 calories). The foods entered can be selected from a comprehensive database of foods that are built into the device. If the device indicates the selected foods in the amounts indicated are appropriate, it can provide a ‘go ahead signal’. Once the foods are consumed, the subject can have an opportunity to adjust the ‘actual’ amount of food consumed. The device might periodically trigger an alarm to let the subject know that it is time to eat a small snack to avoid going into an excessive energy deficit. This procedure of recording foods consumed and getting feedback from the device can repeat itself for 24 hours. The device can communicate with other computers, websites or other platforms or functionalities as can any other devices, systems or processes according to various embodiments of the invention. Software on such a platform can provide a graphical display and printout of the energy surpluses and deficits that occurred during the day. In addition, in this particular variation, the device can link the food codes in the model with food codes in a comprehensive database in the computer, to provide the subject with an in-depth analysis of macro- and micro-nutrient intake, can compare that intake to recommended intakes, and can provide food intake recommendations to correct for nutrient inadequacies.

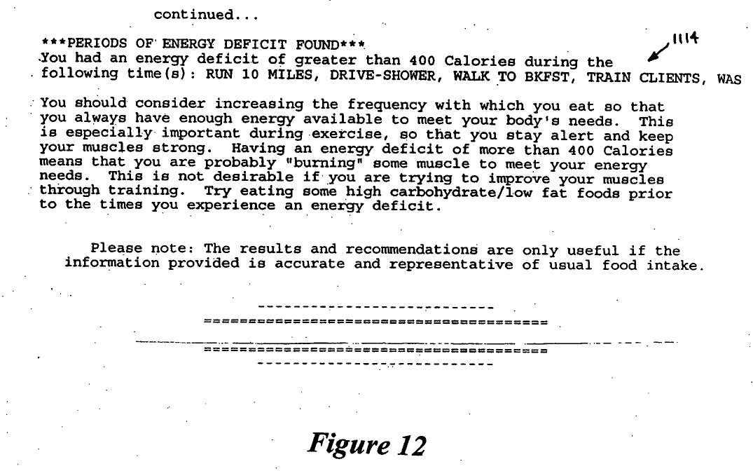

FIGS. 11 and 12 illustrate examples of screenshots for a remote home personal computer (PC) user input display, according to one embodiment of the present invention. The example screenshots shown can be output by a processor associated with a device in accordance with an embodiment of the invention. The information shown is by way of example, and other energy balance-related information can be output or otherwise displayed by devices, systems, and methods in accordance with other embodiments of the invention. For example, the screenshot 1100 shown in FIG. 11 depicts energy balance information associated with a particular user. Line 1102 can provide information related to physical characteristics of the user including, but not limited to, age, weight, height, and resting energy expenditure (REE). Line 1104 can provide information related to energy intake including, but not limited to, calorie distribution, protein component, fat component, carbohydrate component, total Kcal, Kcal requirement for daytime activities, Kcal requirement for evening rest, and a total Kcal requirement. Line 1106 can provide information related to a daily activity routine associated with the user including, but not limited to, a description of the activity, beginning time, end time, and an energy balance calculation associated with each activity and set of times. Line 1108 can provide information related to a total energy intake compared to a predicted requirement, such as “Your total energy intake is 102% of the predicted requirement.” Line 1110 can provide information related to background for a dietary strategy. Line 1112 can provide information related to periods of energy surplus, and recommendations for the particular user, and line 1114 in FIG. 12 can provide information related to periods of energy deficits, and additional recommendations for the particular user, In this manner, the information provided in these screenshots can assist a user to adjust his or her eating habits.

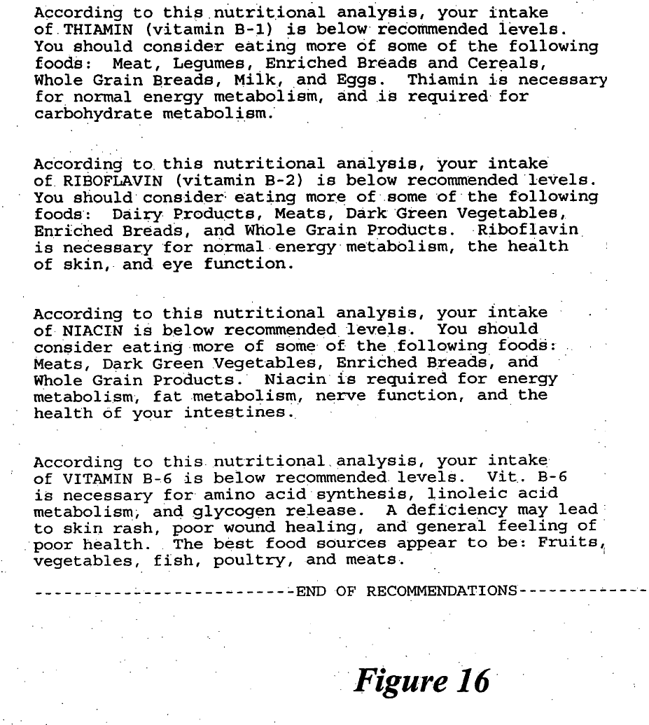

FIGS. 13, 14, 15, and 16 depict examples of screenshots for a daily summary of caloric consumption and associated energy balance. These depictions can provide a user the relative nutrient content of the foods consumed for a particular period. The example screenshots shown can be output by a processor associated with a device in accordance with an embodiment of the invention. The information shown is by way of example, and other energy balance-related information can be output or otherwise displayed by devices, systems, and methods in accordance with other embodiments of the invention.

FIG. 13 depicts an example of a screenshot that displays a more detailed analysis of the relative nutrient content of some or all food consumed by a particular user at a particular time. For example, the screenshot 1300 shown in FIG. 13 depicts detailed nutrient information associated with a particular food or set of foods. Line 1302 can provide information associated with a particular user including, but not limited to, age, days analyzed, foods analyzed, estimated calories required to maintain current weight, and estimated ideal weight. Column 1304 can provide information associated with a particular component or nutrient including, but not limited to, water, energy (Kcal), energy (KJ), protein, carbohydrate, total fat, saturated fat, monosaturated fat, polyunsaturated fat, cholesterol, crude fiber, dietary fiber, ash, calcium, phosphorus, magnesium, iron, zinc, copper, manganese, sodium, potassium, vitamin A (IU), vitamin A(RE), alpha tocoph, total tocoph, thiamin, riboflavin, niacin, vitamin B-6, vitamin B-12, folacin, vitamin C, pantothenate, alcohol, caffeine, and refuse. Line 1306 can include, but is not limited to, actual numeric measurements, recommended amounts, percentage difference greater than or equal to zero, and a graphical display of the percentage difference. Line 1308 can include, but is not limited to, a percentage contribution breakdown, actual and desired, of nutrient groups such as protein, carbohydrate, fat, and related ratios.

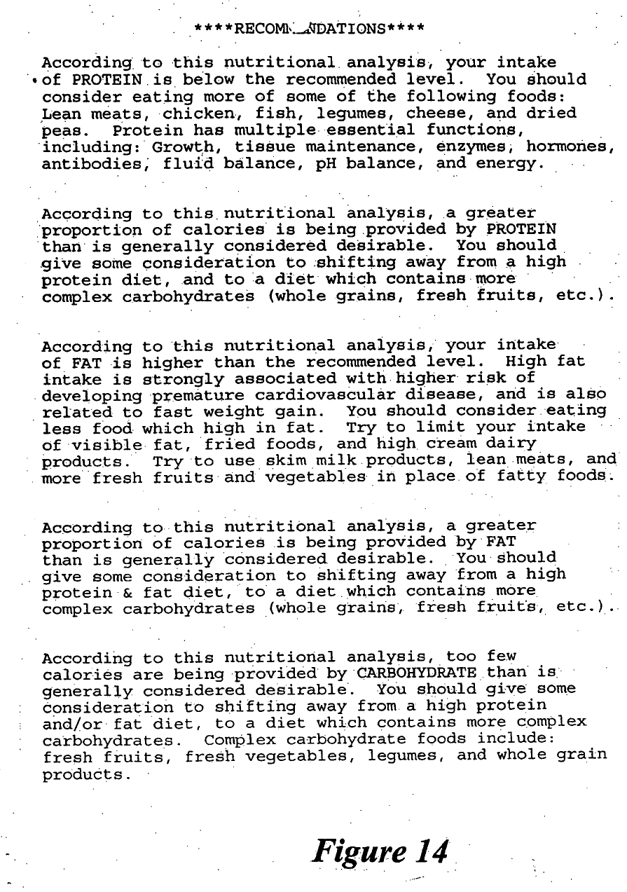

FIGS. 14, 15, and 16 illustrate examples of recommendations associated with the detailed analysis of the relative nutrient content of some or all food consumed by a particular user at a particular time shown in FIG. 13. Such recommendations can be for particular nutrients, components, nutrient groups, ratios, or any other data associated with an energy-balance determination or calculation.

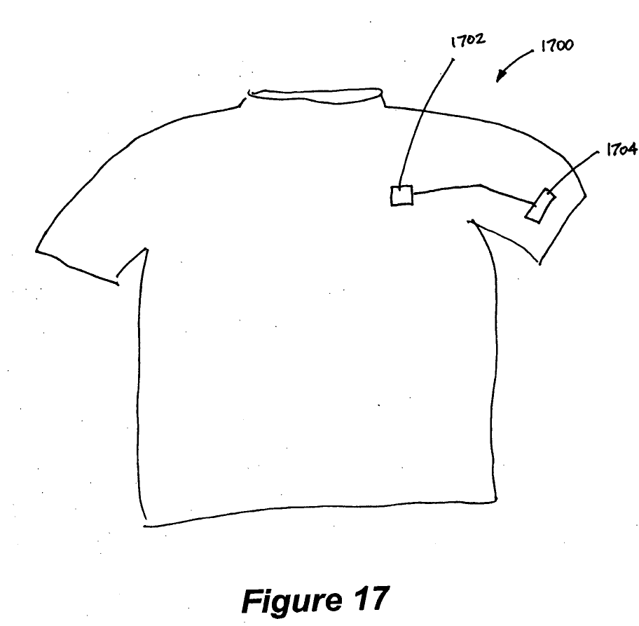

FIG. 17 illustrates another apparatus in accordance with an embodiment of the invention. The apparatus 1700 shown can be adapted to monitor an energy balance deviation associated with a person and capable of being worn by or accompanying the person. The apparatus 1700 can include an input component 1702, and a processor 1704. The input component 1702 can be adapted to receive at least one input associated with energy expenditure of a person, and receive an input associated with energy intake of the person. The processor 1704 can be adapted to calculate an energy balance function based in part on the energy expenditure and the energy intake over a period of time, designate at least one boundary not to be exceeded by said energy balance function, and display information corresponding to said energy balance function and said at least one boundary.

In one embodiment, the apparatus can be incorporated into a wearable article of clothing such as a sportshirt, a shirt, pants, shorts, hat, glasses, or another suitable article. One embodiment includes a sportshirt that can be worn by athletes to train, perform, play sports or participate in other activities, while implementing some or all of the processes described herein.

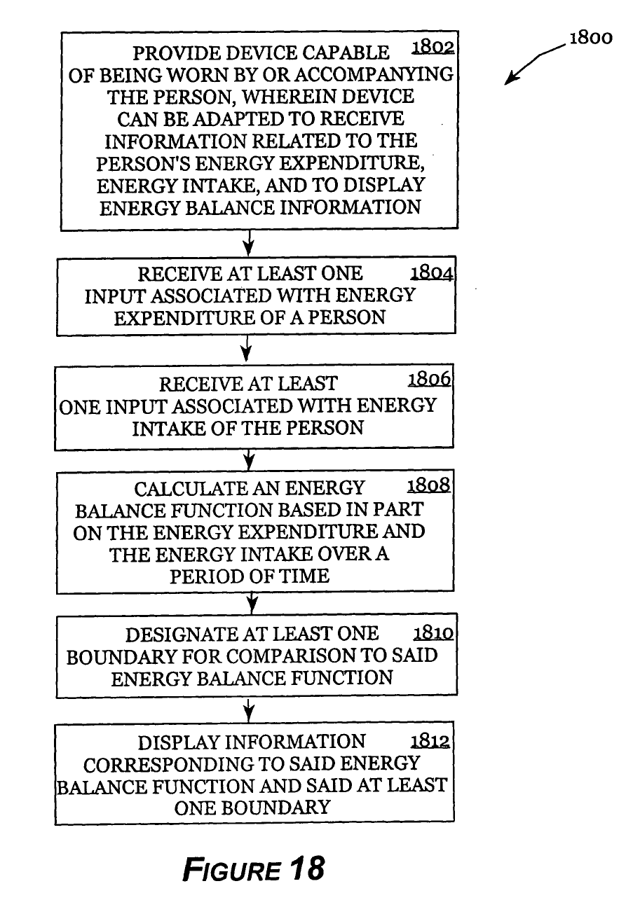

FIG. 18 illustrates a method 1800 that can be implemented by devices, systems, and apparatus in accordance with an embodiment of the invention. The method 1800 is adapted to automatically determine an energy balance deviation associated with a person.

The method 1800 begins at block 1802. At block 1802, a device capable of being worn by or accompanying the person is provided. In the example shown in FIG. 18, the device can be adapted to receive information related to the person's energy expenditure, energy intake, and to display energy balance information.

In one embodiment, receiving at least one input associated with energy expenditure of a person can comprise determining a basal energy expenditure and a work related energy expenditure. In another embodiment, basal energy expenditure can be based at least in part on at least one of the following: a person's gender, weight, and age. In another embodiment, work related energy expenditure can be based at least in part on at least one of the following: a person's body temperature, heart rate, and movement velocity.

Block 1802 is followed by block 1804, in which at least one input associated with energy expenditure of a person is received.

In one embodiment, the at least one input associated with energy expenditure of a person can comprise at least one of the following: a manually entered input, and an automatically measured input. In another embodiment, receiving an input associated with energy intake of the person can comprise a manual selection of a food item consumed by the person. In another embodiment, receiving an input associated with energy intake of the person can comprise determining a caloric value for a food item consumed by the person.

Block 1804 is followed by block 1806, in which at least one input associated with energy intake of the person is received.

Block 1806 is followed by block 1808, in which an energy balance function based in part on the energy expenditure and the energy intake over a period of time is calculated.

In one embodiment, calculating an energy balance function based in part on the energy expenditure and the energy intake over a period of time can comprise determining an instantaneous energy balance function. In another embodiment, calculating an energy balance function based in part on the energy expenditure and the energy intake over a period of time can comprise determining an energy balance function at a predefined amount of time. In yet another embodiment, predefined amount of time can comprise at least one of the following: a minute, 15 minutes, and 60 minutes. In another embodiment, calculating an energy balance function based in part on the energy expenditure and the energy intake over a period of time can comprise determining a difference between the energy expenditure and energy intake associated with the person. Moreover, in another embodiment, calculating an energy balance function based in part on the energy expenditure and the energy intake over a period of time can comprise determining a ratio between the energy expenditure and energy intake associated with the person.

Block 1808 is followed by block 1810, in which at least one boundary for comparison to said energy balance function is designated.

In one embodiment, designating at least one boundary not to be exceeded by said energy balance function can comprise at least one of the following: designating one boundary, and designating two boundaries. In another embodiment, designating at least one boundary not to be exceeded by said energy balance function can comprise at least one of the following: designating a high boundary, and designating a low boundary. In yet another embodiment, designating at least one boundary not to be exceeded by said energy balance function can comprise at least one of the following: manually designating at least one boundary, and automatically designating at least one boundary.

Block 1810 is followed by block 1812, in which information corresponding to said energy balance function and said at least one boundary is displayed. The method 1800 ends at block 1812.

Another embodiment of a method can include providing a notification when said energy balance function exceeds said at least one boundary. Yet another embodiment of a method can include providing a notification when said energy balance function will be exceeded upon additional energy intake. Yet another method can include providing notification when, based on energy balance function information, the person needs additional energy intake.

Still another embodiment can include loading stored information relating to energy intake, energy expenditure, energy balance function and boundaries to a remote platform. In one embodiment, loading stored information relating to energy intake, energy expenditure, energy balance function and boundaries to a remote platform comprises transmitting the information from the remote platform through a wireless medium. In another embodiment, loading stored information relating to energy intake, energy expenditure, energy balance function and boundaries to a remote platform comprises transmitting the information from the remote platform through a physical connection.

Still another embodiment of the method includes a device that can device comprise at least one button adapted to permit input associated with energy expenditure of a person, and at least one button adapted to permit input associated with energy intake of a person. In another embodiment, the device can comprise at least one button adapted to permit input associated with energy expenditure of a person, and at least one button adapted to permit input associated with energy intake of a person.

While the above description contains many specifics, these specifics should not be construed as limitations on the scope of the invention, but merely as exemplifications of the disclosed embodiments. Those skilled in the art will envision any other possible variations that are within the scope of the invention.

1. A method for automatically determining an energy balance deviation associated with a person, comprising:
providing a device capable of being worn by or accompanying the person, the device adapted to receive information related to the person's energy expenditure, energy intake, and to display energy balance information;
receiving at least one input associated with energy expenditure of a person;
receiving at least one input associated with energy intake of the person;
calculating an energy balance function based in part on the energy expenditure and the energy intake over a period of time;
designating at least one boundary for comparison to said energy balance function; and
displaying information corresponding to said energy balance function and said at least one boundary.

2. The method of claim 1, wherein receiving at least one input associated with energy expenditure of a person comprises determining a basal energy expenditure and a work related energy expenditure.

3. The method of claim 2, wherein the basal energy expenditure is based at least in part on at least one of the following: a person's gender, weight, and age.

4. The method of claim 2, wherein the work related energy expenditure is based at least in part on at least one of the following: a person's body temperature, heart rate, and movement velocity.

5. The method of claim 1, wherein the at least one input associated with energy expenditure of a person comprises at least one of the following: a manually entered input, and an automatically measured input.

6. The method of claim 1, wherein receiving an input associated with energy intake of the person comprises a manual selection of a food item consumed by the person.

7. The method of claim 1, wherein receiving an input associated with energy intake of the person comprises determining a caloric value for a food item consumed by the person.

8. The method of claim 1, wherein calculating an energy balance function based in part on the energy expenditure and the energy intake over a period of time comprises determining an instantaneous energy balance function.

9. The method of claim 1, wherein calculating an energy balance function based in part on the energy expenditure and the energy intake over a period of time comprises determining an energy balance function at a predefined amount of time.

10. The method of claim 9, wherein the predefined amount of time comprises at least one of the following: a minute, 15 minutes, 60 minutes.

11. The method of claim 1, wherein calculating an energy balance function based in part on the energy expenditure and the energy intake over a period of time comprises determining a difference between the energy expenditure and energy intake associated with the person.

12. The method of claim 1, wherein calculating an energy balance function based in part on the energy expenditure and the energy intake over a period of time comprises determining a ratio between the energy expenditure and energy intake associated with the person.

13. The method of claim 1, wherein designating at least one boundary not to be exceeded by said energy balance function comprises at least one of the following: designating one boundary, and designating two boundaries.

14. The method of claim 1, wherein designating at least one boundary not to be exceeded by said energy balance function comprises at least one of the following: designating a high boundary, and designating a low boundary.

15. The method of claim 1, wherein designating at least one boundary not to be exceeded by said energy balance function comprises at least one of the following: manually designating at least one boundary, automatically designating at least one boundary.

16. The method of claim 1, further comprising:
providing a notification when said energy balance function exceeds said at least one boundary.

17. The method of claim 1, further comprising:
providing a notification when said energy balance function will be exceeded upon additional energy intake.

18. The method of claim 1, further comprising:
providing notification when, based on energy balance function information, the person needs additional energy intake.

19. The method of claim 1, further comprising:
loading stored information relating to energy intake, energy expenditure, energy balance function and boundaries to a remote platform.

20. The method of claim 19, wherein loading stored information relating to energy intake, energy expenditure, energy balance function and boundaries to a remote platform comprises transmitting the information from the remote platform through a wireless medium.

21. The method of claim 19, wherein loading stored information relating to energy intake, energy expenditure, energy balance function and boundaries to a remote platform comprises transmitting the information from the remote platform through a physical connection.

22. The method of claim 1, wherein the device comprises at least one button adapted to permit input associated with energy expenditure of a person, and at least one button adapted to permit input associated with energy intake of a person.

23. The method of claim 1, wherein the device comprises at least one transducer adapted to permit input associated with energy expenditure of a person, and at least one transducer adapted to permit input associated with energy intake of a person.

24. An apparatus for monitoring an energy balance deviation associated with a person and capable of being worn by or accompanying the person, comprising:
an input component adapted to
receive at least one input associated with energy expenditure of a person;
receive an input associated with energy intake of the person;
a processor adapted to
calculate an energy balance function based in part on the energy expenditure and the energy intake over a period of time;
designate at least one boundary not to be exceeded by said energy balance function; and
display information corresponding to said energy balance function and said at least one boundary.

25. The apparatus of claim 24, wherein the element receive at least one input associated with energy expenditure of a person comprises determine a basal energy expenditure and a work related energy expenditure.

26. The apparatus of claim 25, wherein the basal energy expenditure is based at least in part on at least one of the following: a person's gender, weight, and age.

27. The apparatus of claim 25, wherein the work related energy expenditure is based at least in part on at least one of the following: a person's body temperature, heart rate, and movement velocity.

28. The apparatus of claim 24, wherein the at least one input associated with energy expenditure of a person comprises at least one of the following: a manually entered input, and an automatically measured input.

29. The apparatus of claim 24, wherein the element receive an input associated with energy intake of the person comprises receive a manual selection of a food item consumed by the person.

30. The apparatus of claim 24, wherein the element receive an input associated with energy intake of the person comprises determine a caloric value for a food item consumed by the person.

31. The apparatus of claim 24, wherein the element calculate an energy balance function based in part on the energy expenditure and the energy intake over a period of time comprises determine an instantaneous energy balance function.

32. The apparatus of claim 24, wherein the element calculate an energy balance function based in part on the energy expenditure and the energy intake over a period of time comprises determine an energy balance function at a predefined amount of time.

33. The apparatus of claim 32, wherein the predefined amount of time comprises at least one of the following: a minute, 15 minutes, 60 minutes.

34. The apparatus of claim 24, wherein the element calculate an energy balance function based in part on the energy expenditure and the energy intake over a period of time comprises determine a difference between the energy expenditure and energy intake associated with the person.

35. The apparatus of claim 24, wherein the element calculate an energy balance function based in part on the energy expenditure and the energy intake over a period of time comprises determine a ratio between the energy expenditure and energy intake associated with the person.

36. The apparatus of claim 24, wherein the element designate at least one boundary not to be exceeded by said energy balance function comprises at least one of the following: designate one boundary, and designate two boundaries.

37. The apparatus of claim 24, wherein the element designate at least one boundary not to be exceeded by said energy balance function comprises at least one of the following: designate a high boundary, and designate a low boundary.

38. The apparatus of claim 24, wherein the element designate at least one boundary not to be exceeded by said energy balance function comprises at least one of the following: manually designate at least one boundary, automatically designate at least one boundary.

39. The apparatus of claim 24, wherein the processor is further adapted to:
provide a notification when said energy balance function exceeds said at least one boundary.

40. The apparatus of claim 39, wherein the processor is further adapted to:
provide a notification when said energy balance function will be exceeded upon additional energy intake.

41. The apparatus of claim 39, wherein the processor is further adapted to:
provide notification when, based on energy balance function information, the person needs additional energy intake.

42. The apparatus of claim 39, wherein the processor is further adapted to:
load stored information relating to energy intake, energy expenditure, energy balance function and boundaries to a remote platform.

43. The apparatus of claim 42, wherein the element load stored information relating to energy intake, energy expenditure, energy balance function and boundaries to a remote platform comprises transmit the information from the remote platform through a wireless medium.

44. The apparatus of claim 42, wherein the element load stored information relating to energy intake, energy expenditure, energy balance function and boundaries to a remote platform comprises transmit the information from the remote platform through a physical connection.

45. The apparatus of claim 24, wherein the device comprises at least one button adapted to permit input associated with energy expenditure of a person, and at least one button adapted to permit input associated with energy intake of a person.

46. The apparatus of claim 24, wherein the device comprises at least one transducer adapted to permit input associated with energy expenditure of a person, and at least one transducer adapted to permit input associated with energy intake of a person.

47. A computer readable medium containing program code for automatically determining an energy balance deviation associated with a person, comprising:
program code adapted to provide a device capable of being worn by or accompanying the person to
receive information related to the person's energy expenditure, energy intake, and to display energy balance information;
receive at least one input associated with energy expenditure of a person;
receive at least one input associated with energy intake of the person;
calculate an energy balance function based in part on the energy expenditure and the energy intake over a period of time;
designate at least one boundary for comparison to said energy balance function; and
display information corresponding to said energy balance function and said at least one boundary.

48. The computer readable medium of claim 47, wherein program code adapted to receive at least one input associated with energy expenditure of a person comprises program code adapted to determine a basal energy expenditure and a work related energy expenditure.

49. The computer readable medium of claim 48, wherein the basal energy expenditure is based at least in part on at least one of the following: a person's gender, weight, and age.

50. The computer readable medium of claim 48, wherein the work related energy expenditure is based at least in part on at least one of the following: a person's body temperature, heart rate, and movement velocity.

51. The computer readable medium of claim 47, wherein the at least one input associated with energy expenditure of a person comprises at least one of the following: a manually entered input, and an automatically measured input.

52. The computer readable medium of claim 47, wherein program code adapted to receive an input associated with energy intake of the person comprises a manual selection of a food item consumed by the person.

53. The computer readable medium of claim 47, wherein program code adapted to receive an input associated with energy intake of the person comprises program code adapted to determine a caloric value for a food item consumed by the person.

54. The computer readable medium of claim 47, wherein program code adapted to calculate an energy balance function based in part on the energy expenditure and the energy intake over a period of time comprises program code adapted to determine an instantaneous energy balance function.

55. The computer readable medium of claim 47, wherein program code adapted to calculate an energy balance function based in part on the energy expenditure and the energy intake over a period of time comprises program code adapted to determine an energy balance function at a predefined amount of time.

56. The computer readable medium of claim 55, wherein the predefined amount of time comprises at least one of the following: a minute, 15 minutes, 60 minutes.

57. The computer readable medium of claim 47, wherein program code adapted to calculate an energy balance function based in part on the energy expenditure and the energy intake over a period of time comprises program code adapted to determine a difference between the energy expenditure and energy intake associated with the person.

58. The computer readable medium of claim 47, wherein program code adapted to calculate an energy balance function based in part on the energy expenditure and the energy intake over a period of time comprises program code adapted to determine a ratio between the energy expenditure and energy intake associated with the person.

59. The computer readable medium of claim 47, wherein program code adapted to designate at least one boundary not to be exceeded by said energy balance function comprises at least one of the following: program code adapted to designate one boundary, and program code adapted to designate two boundaries.

60. The computer readable medium of claim 47, wherein program code adapted to designate at least one boundary not to be exceeded by said energy balance function comprises at least one of the following: program code adapted to designate a high boundary, and program code adapted to designate a low boundary.

61. The computer readable medium of claim 47, wherein program code adapted to designate at least one boundary not to be exceeded by said energy balance function comprises at least one of the following: program code adapted to permit a manually designation of at least one boundary, program code adapted to automatically designate at least one boundary.

62. The computer readable medium of claim 47, further comprising:
program code adapted to provide a notification when said energy balance function exceeds said at least one boundary.

63. The computer readable medium of claim 47, further comprising:
program code adapted to provide a notification when said energy balance function will be exceeded upon additional energy intake.

64. The computer readable medium of claim 47, further comprising:
program code adapted to provide notification when, based on energy balance function information, the person needs additional energy intake.

65. The computer readable medium of claim 47, further comprising:
program code adapted to load stored information relating to energy intake, energy expenditure, energy balance function and boundaries to a remote platform.

66. The computer readable medium of claim 65, wherein program code adapted to load stored information relating to energy intake, energy expenditure, energy balance function and boundaries to a remote platform comprises program code adapted to transmit the information from the remote platform through a wireless medium.

67. The computer readable medium of claim 65, wherein program code adapted to load stored information relating to energy intake, energy expenditure, energy balance function and boundaries to a remote platform comprises program code adapted to transmit the information from the remote platform through a physical connection.

68. The computer readable medium of claim 47, wherein the device comprises at least one button adapted to permit input associated with energy expenditure of a person, and at least one button adapted to permit input associated with energy intake of a person.

69. The computer readable medium of claim 47, wherein the device comprises at least one transducer adapted to permit input associated with energy expenditure of a person, and at least one transducer adapted to permit input associated with energy intake of a person.
